(19) United States
(12) Patent Application Publication
(10) Pub. No.: US 2023/0338334 A1
(43) Pub. Date: Oct. 26, 2023

(54) A PHARMACEUTICAL FORMULATION TO TREAT ACUTE AND CHRONIC PANCREATITIS

(71) Applicant: CEDARS-SINAI MEDICAL CENTER, Los Angeles, CA (US)

(72) Inventors: Rajiv Manharbhai Mehta, Surat (IN); Nilesh Vipinchandra Mehta, Mumbai (IN); Stephen Pandol, Los Angeles, CA (US)

(21) Appl. No.: 18/026,763

(22) PCT Filed: Sep. 10, 2021
(86) PCT No.: PCT/IN2021/050889

§ 371 Date: Mar. 16, 2023

(30) Foreign Application Priority Data
Sep. 26, 2020 (IN) 202021041898

(51) Int. Cl. A61K 31/366; A61K 31/40; A61K 31/22; A61K 31/505; A61K 31/4418; A61K 31/47; A61K 31/405; A61K 31/198; A61K 31/436; A61K 31/7034; A61K 31/4709

(52) U.S. Cl. CPC A61K 31/366; A61K 31/40; A61K 31/22; A61K 31/505; A61K 31/4418; A61K 31/47; A61K 31/405; A61K 31/198; A61K 31/436; A61K 31/7034; A61K 31/4709

(57) ABSTRACT

A formulation treating acute and chronic pancreatitis in patients. A pharmaceutical composition of statin and antifibrotic agent. The stating and the antifibrotic agent mixed in appropriate excipient to make acceptable as pharmaceutical formulation by mammal. The pharmaceutical formulation is adapted as oral in one ore more doses to inhibit reoccurrence of acute or chronic pancreatitis.

### FIELD ON INVENTION

The present subject matter of invention is generally to a formulation treating acute and chronic pancreatitis in patients.

### BACKGROUND OF INVENTION

Pancreatic cancer is the one of most lethal malignant neoplasm with extremely lower mortality rate with life expectancy of less than 5 years.

Extensive research has identified that chronic pancreatitis (CP) is one of the strongest identified risk factor for pancreatic cancer and increases the risk by 13.3 folds.

CP is progressive inflammatory condition that is believed to rise from repeated overt or silent episodes of Acute Pancreatitis (AP).

Generally, Acute pancreatitis is an inflammation of the pancreas that occurs when digestive enzymes leak out of the pancreatic ducts and damage the pancreas. The etiology of acute pancreatitis is unknown. It is a syndrome, which may result from multiple factors. The common feature to all cases of acute pancreatitis is a release and activation of digestive enzymes within the exocrine pancreas gland causing inflammation, injury, autolysis and necrosis to the organ. This can also lead to hemorrhage and pseudocyst formation within the gland. Severe upper abdominal pain, nausea and vomiting are the most common symptoms.

As a result of AP and repeated damage to the endocrine and exocrine cells of the pancreas, the disease leads to clinical manifestations with exocrine and endocrine insufficiencies.

However, till the date there is no treatments are available to reduce the repeated attacks of acute inflammation of pancreas in recurrent AP and CP. Hence, interventions that can prevent the development of pancreatic cancer in high risk populations would therefore address a major public health problem and would have a high clinical impact.

Therefore, there is a need in the art to develop a formulation which can effectively treat AP and CP patients.

### DETAILED DESCRIPTION OF THE INVENTION

Before describing in detail embodiments that are in accordance with the invention, it should be observed that the embodiments reside primarily in a AP and CP treatment. Accordingly, the components have been described to include only those specific details that are pertinent to understanding the embodiments of the invention so as not to obscure the disclosure with details that will be readily apparent to those of ordinary skill in the art having the benefit of the description herein.

In this document, the terms “comprises”, “comprising” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Further, before describing in detail embodiments that are in accordance with the invention, it should be observed that all the scientific and technical terms used herein for describing the invention have same meanings as would be understood by a person skilled in the art.

Generally speaking, pursuant to various embodiments, a formulation which treats AP and CP patients.

Examples of constructions, materials, dimensions and manufacturing processes are provided for selected elements. All other elements employ that which is known to those skilled in the field of the invention. Those skilled in the art will recognize that many of the examples provided have suitable alternatives which may also be utilized.

The present invention discloses methods and compositions useful for the treatment of acute pancreatitis.

In an embodiment of the present invention, disclosed is a composition comprising a statin. Statin is at least one ring-opened 7-substituted-3,5-dihydroxyheptanoic acid or ring-opened 7-substituted-3,5-dihydroxyheptenoic acid.

Preferably the statin is selected from atorvastatin, lovastatin, pravastatin, simvastatin, rosuvastatin, mevastatin, cerivastatin, itavastatin, fluvastatin, pitavastatin, wuxistatin and salts thereof.

In yet another embodiment, the antifibrotic agent is selected from groups of carotenoids, flavonoids, isoflavonoids, hydroxyacids, phenols, polyphenols, sulphite salts, terpene alcohols, thiourea and flavonoids, with proven antioxidant, antifibrotic, or anti-carcinogenic properties.

In one more embodiment of invention, antifibrotic agent is selected from N-acetylcysteine, pirfenidone, tacrolimus, tannic acid and galunisertib.

In one more embodiment, the preferred dosage forms of the pharmaceutical compositions of the present invention are formulated in formulation (hereafter referred as Pharmaceutical Formulation) as solid dosage forms adapted for oral administration. Tablet dosage forms are the particularly preferred solid dosage forms of the stabilized pharmaceutical compositions of the present invention.

In still one more embodiment, the tablet dosage forms may contain for example, as excipients, any pharmaceutically acceptable binder, disintegrant, diluent, carrier, preservative or combination thereof. For the purpose of oral preparations of the present invention, pharmaceutically acceptable inert carriers or diluent or filler can be either solid or not. Among other preferred dosage forms useful for formulating the stabilized pharmaceutical compositions of the present invention include powders, dispersible granules, capsules and cachets.

In yet one more embodiment, the pharmaceutical formulation of the present invention may also contain any pharmaceutically acceptable excipient or combination thereof. Conventional pharmaceutical excipients include those which function in a dosage form, for example, as a diluent, binder, disintegrant, carrier, colorant, preservative or coating material.

Examples of pharmaceutically acceptable excipients include, but are not limited to, lactose, sugar, saccharose, corn starch, hydrolyzed starch (malto-dextrine), modified corn starch, microcrystalline cellulose, croscarmellose sodium, polyvinylpyrrolidone, polyvinylalcohol, hydroxypropylcellulose, hydroxymethylcellulose, hydroxypropylmethylcellulose, methylcellulose, ethylcellulose, gelatin, cross-linked PVP, sodium carboxymethyl starch, magnesium stearate, sodium stearyl fumarate, polyethylene glycol, stearic acid, beta cyclodextrin and mixtures thereof.

In a preferred process comprises the steps of preparing granules of statin and antifibrotic agents together with the methacrylic polymer, and then compressing said granules with extra-granular excipients, into a tablet.

Granules manufacture can take place according to various techniques, such fluidized bed technology, tank mixing, or crushing compacts. Granules are optionally, but not necessarily, compressed into tablets.

One embodiment includes the steps of dry mixing the components, compressing into a compressed form and crushing said form into granules.

One more embodiment specifying pharmaceutical formulation for use in mammal for atleast once twice or thrice in form of granules of tablets mixed to prepare final dose in 40 mg simvastatin, 2000 mg of N-acetylcysteine and appropriate excipient.

In another embodiment, the pharmaceutical formulation is mixed with long release drug carrier or excipient to give drug effect for at least 24 hours to avoid multiple doses.

Yet one more pharmaceutical formulation 10 and 50 wt % of the excipient is mixed in proportion between 2 and 18 wt % the statin and between 30-50% the antifibrotic agent to make

Those skilled in the art will realize that the above-recognized advantages and other advantages described herein are merely exemplary and are not meant to be a complete rendering of all of the advantages of the various embodiments of the invention.

Other features such as variation of drug dose and drug delivery method may also be used to treat AP and CP. For the purpose of the following discussion, the exemplary embodiments are directed to a formulation which is particularly suitable for AP and CP treatment. However, with simple modifications in construction in preparing various salts, various analogs of the present invention may be used for other medical applications not fully discussed herein.

In the foregoing provisional specification, specific embodiments of the invention have been described. However, one of ordinary skill in the art appreciates that various modifications and changes can be made to the invention without deviating from the scope of the invention. Accordingly, the provisional specification is to be regarded in an illustrative rather than a restrictive sense, and all such modifications are intended to be included within the scope of the invention.

We claim:

1. A composition for the treatment of acute pancreatitis and chronic pancreatitis in a mammal comprising:
a first compound comprising a statin, and
a second compound comprising an antifibrotic agent.

2. The composition of claim 1 wherein the statin is selected from atorvastatin, lovastatin, pravastatin, simvastatin, rosuvastatin, mevastatin, cerivastatin, itavastatin, fluvastatin, pitavastatin, wuxistatin and salts thereof.

3. The composition of claim 1 wherein, statin is at least one ring-opened 7-substituted-3,5-dihydroxyheptanoic acid or ring-opened 7-substituted-3,5-dihydroxyheptenoic acid.

4. The composition of claim 1 wherein the antifibrotic agent is selected from N-acetylcysteine, pirfenidone, tacrolimus, tannic acid and galunisertib.

5. The composition of claim 1 wherein the statin is simvastatin or salt thereof and antifibrotic agent is N-acetylcysteine.

6. The composition of claim 2 wherein, the statin plurality dose is ranging from 10 mg to 80 mg.

7. The composition of claim 4 wherein, plurality dose of antifibrotic agent is selected from 400 mg to 4000 mg.

8. A dispersed solid dose formulation for the treatment of acute pancreatitis and chronic pancreatitis in a mammal comprising:
a statin or salt thereof,
an antioxidant composition, and
at least one excipient.

9. The composition of claim 8 wherein solid dose formation comprising between 10 and 50 wt % of the excipient, between 2 and 18 wt % the statin and between 30-50% the antifibrotic agent.

10. The composition of claim 8 wherein dose is selected from once daily, twice daily and thrice daily.
